(19) United States
(12) Patent Application Publication
(10) Pub. No.: US 2007/0251523 A1
(43) Pub. Date: Nov. 1, 2007

(54) Gas-Treatment Devices

(76) Inventor: Christophe Van Landuyt, Louveciennes (FR)

Correspondence Address: LOUIS WOO;LAW OFFICE OF LOUIS WOO, 717 NORTH FAYETTE STREET, ALEXANDRIA, VA, 22314, US

(21) Appl. No.: 10/579,200

(22) PCT Filed: Nov. 30, 2004
(86) PCT No.: PCT/GB04/05039

§ 371 Date: Feb. 1, 2007

(30) Foreign Application Priority Data
Dec. 16, 2003 (GB) 0329054.1

(51) Int. Cl. A61M 16/10

(52) U.S. Cl. 128/203.12

(57) ABSTRACT

An HME device (2, 2′) for a tracheostomy tube (1) has a housing (20, 20′) with an opening (23), which can be connected to the machine end (15) of the tube. A cylindrical HME unit (30, 30) with an HME element (32 and 33) at opposite ends and a blocking plate (34) midway is rotatable within the housing (20). In normal use, the blocking plate (34) is out of alignment with the opening (23) so that gas can flow along the tube (1) through the device (2, 2′). When the patient wishes to speak, he twists the HME unit (30) to align the blocking plate (34) with the opening (23) to block flow and thereby prevent gas emerging from the machine end (15) of the p tube (1) so that it is diverted to the larynx via fenestrations (13) in the side of the tube.

This invention relates to gas-treatment devices of the kind for use with a tracheostomy tube, the device including a housing adapted for connection to the tube, a gas passage in the housing and a gas-treatment unit mounted with the housing.

The invention is more particularly concerned with heat and moisture exchangers (HMEs) or the like for use with tracheostomy tubes.

In normal breathing, inhaled air passes through the nose where it is warmed and moistened before passing to the trachea and bronchial passages. Where a patient breathes via a tracheal tube or laryngeal mask, gas is supplied directly to the trachea, by-passing the nose. The gas is, therefore, preferably warmed and moistened to prevent discomfort and damage to the lining of the trachea. This is often achieved by a heat and moisture exchange device (HME) connected to the tracheal tube to receive both exhaled and inhaled gases. The HME has a moisture-absorbing element, such as a treated paper element or a foam, which absorbs moisture in exhaled gases and transfers a major part of this to the inhaled gases. The element also warms inhaled gas in the same way. HMEs are sold by Portex Limited of Hythe, England under the trade mark Thermovent Examples of HMEs are described in: GB 2303307; GB 2321600; GB 2277689; GB 2268496; GB2267840; GB 2233904; EP 535016; EP 533644; EP 387220; EP 265163; EP 413127; U.S. Pat. No. 4,516,573; U.S. Pat. No. 4,090,513; U.S. Pat. No. 4,771,770; U.S. Pat. No. 4,200,094; and U.S. Pat. No. 4,048,993. The HME may also include a filter for removing particles, bacteria and viruses from gas supplied to or from the patient.

The patient may have a tracheostomy tube that enables speech. Such tubes have openings or fenestrations above the sealing cuff to allow air from the lungs to pass to the vocal folds. The tube also needs to be fitted with a valve that can be closed to prevent air escaping from the machine end of the tube so that it is diverted to the fenestrations when speech is required. It is also desirable to be able to provide a speech function when the tube is fitted with an HME. In one HME, the Atox Provox Trachphone, the user can block the opening momentarily by pressing down a lid that holds the suction valve.

It is an object of the present invention to provide an alternative gas-treatment device and assembly.

According to one aspect of the present invention there is provided a gas-treatment device of the above-specified kind, characterised in that the gas-treatment unit is displaceable from a first position in which gas can flow from the tube through the gas-treatment unit via the gas passage, to a second position in which the gas passage is substantially blocked preventing flow of gas out of the machine end of the tracheostomy tube.

The gas-treatment unit is preferably displaceable by rotation. The gas-treatment unit may remain in the first or second position unless manually displaced. The gas-treatment unit may include a projection accessible at an end of the housing by which the gas-treatment unit can be displaced. Alternatively, the device may include a resilient member arranged to displace the gas-treatment unit to the first position when released. The gas-treatment unit is preferably cylindrical and includes an HME element, preferably an element at opposite ends. The or each HME element may be of a treated paper.

According to another aspect of the present invention there is provided a tracheostomy tube assembly including a tracheostomy tube and a gas-treatment device according to the above one aspect of the present invention connected at the machine end of the tube.

A tracheostomy tube assembly including an HME, in accordance with the present invention, will now be described, by way of example, with reference to the accompanying drawing, in which:

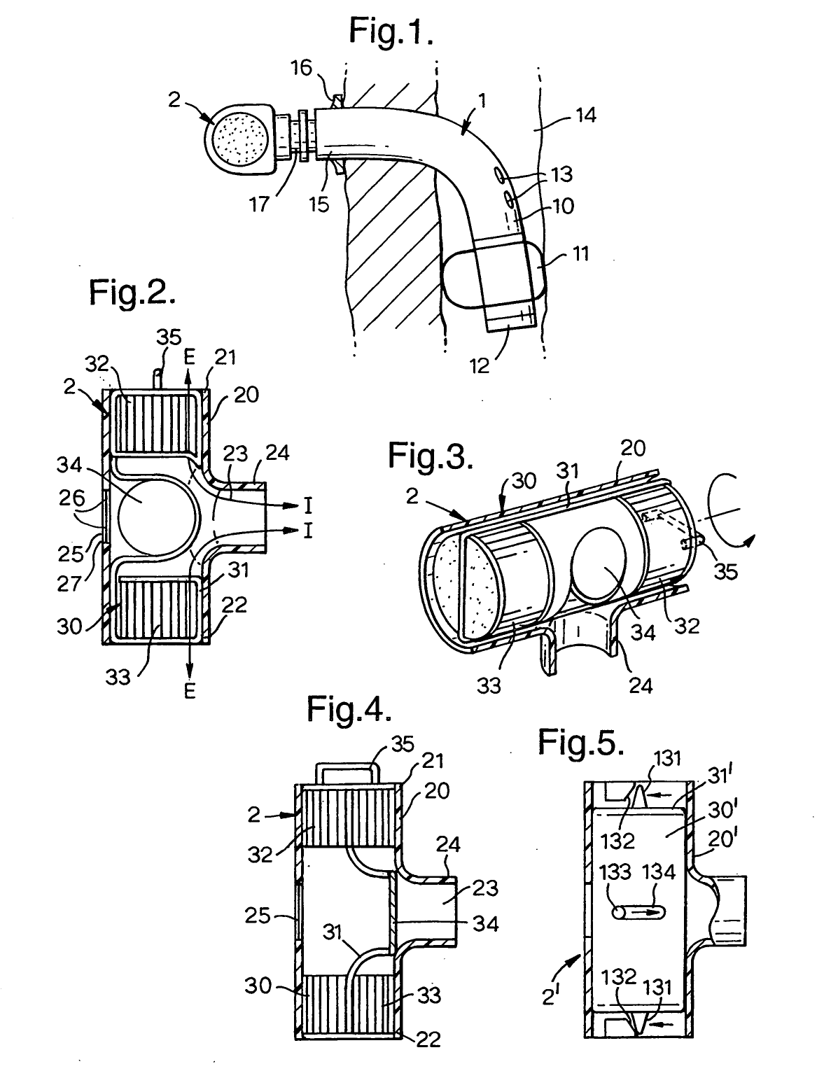

FIG. 1 is a side elevation view of the assembly,

FIG. 2 is a cross-sectional plan view of the HME in an open state;

FIG. 3 is a perspective view of the HME in an open state;

FIG. 4 is a cross-sectional plan view of the HME in a closed, speaking state; and

FIG. 5 is a side elevation view of a modified HME.

With reference first to FIG. 1 the assembly comprises a tracheotomy tube 1 and an HME gas-treatment device 2 connected to the rear, machine end of the tube. The HME device 2 can be switched between an open state when it allows passage of air in and out of the tube 1 and a closed state when it prevents gas emerging from the rear end of the tube.

The tracheostomy tube 1 is conventional, including a curved shaft 10 with a sealing cuff 11 adjacent its forward, patient end 12. The shaft 10 has several fenestrations or openings 13 formed through its wall above the cuff 11 on the outside curve of the shaft. The forward part of the shaft 10 including the fenestrations 13 is positioned within the trachea 14, with the cuff 11 inflated to seal with the surface of the trachea. The rear, machine end 15 of the tube 1 has a flange 16, by which the tube is secured with the patient's neck, and a coupling 17 onto which the HME device 2 is fitted.

With reference now to FIGS. 2 to 4, the HME device 2 is of a cylindrical shape and is oriented to extend transversely of the rear end of the tube 1 and transversely of the axis of the trachea 14. The device 2 has a cylindrical outer housing 20 of a moulded plastics material, which is open at both ends 21 and 22. Midway along its length the housing 20 has a circular opening or passage 23 within a short, outwardly-projecting collar 24 shaped to fit onto the tracheostomy tube coupling 17. Diametrically opposite the opening 23 the housing 20 has a valved suctioning port 25, which may be of any conventional kind, but typically comprises three resilient leaves 26 of segmented shape attached at their outside edge within a circular aperture 27 in the housing. The edges of leaves 26 normally abut one another substantially to seal the aperture 27 but can be opened by pushing a suction catheter or the like through the centre of the aperture to separate the tips of the leaves.

The HME device 2 contains an HME unit 30, which is also substantially cylindrical. The element 30 has a wire support frame 31 retaining two HME elements 32 and 33, one at each end. The HME elements 32 and 33 are of a paper treated with a hygroscopic material to enhance the absorption of moisture. The paper is preferably in strip form with lateral corrugations rolled into a coil so that the corrugations form multiple parallel gas passages extending parallel to the axis. Alternative HME materials could be used, such as foamed plastics. The frame 31 also supports a blocking member 34 in the form of an impermeable circular sheet or plate having a diameter slightly greater than that of the opening 23. The blocking member 34 extends a part way around the circumference of the frame 31 and is located midway along its length, at the same position as the opening 23 and the suctioning port 25. At one end of the HME unit 30, the wire frame 31 is formed into an axially-projecting flange 35, which protrudes from and is accessible at one end of the housing 20 so as to provides a finger grip. The flange 35 is preferably removable so that, when removed, the HME unit 30 cannot be moved out of its open position. The HME unit could take various alternative forms, for example, the frame could be of a plastics material or it could be provided by stiffened paper. The blocking member could be a one-way valve that allows gas to flow into the patient but prevents exhalation from the machine end 15 of the tracheostomy tube 1. It is not necessary for the blocking member to block flow of air totally, only sufficient to divert enough to enable speech.

The HME unit 30 is rotatable about its axis within the housing 20 and is a close sliding fit so that it remains in whatever angular position to which it is moved. The inside of the housing 20 is formed with stops (not shown) that cooperate with the frame 31 to limit rotation of the unit to 90°. In its normal, open position, as shown in FIGS. 2 and 3, the HME unit 30 is positioned so that the blocking member 34 is displaced 90° from the patient opening 23. Air can be exhaled by the patient via the opening 23, flowing outwardly through the two HME elements 32 and 33, and giving up the major part of its humidity and warmth to the elements, as shown by the arrow heads marked “E” (exhalation) in FIG. 2. The patient can also inhale through the device 2 when air flows in the opposite direction indicated by the arrow heads marked “T” (inhalation) in FIG. 2. Air flowing in this direction takes up the majority of the moisture and heat stored in the HME elements 32 and 33 so that the air flowing to the tube 1 is warmed and moistened. With the HME unit 30 in this position, the tracheostomy tube 1 can be suctioned by inserting a suction catheter (not shown) through the suctioning port 25, through the open structure of the frame 31, through the opening 23 and along the tracheostomy tube. When the patient wishes to talk he grips the flange 35 and rotates the HME unit 30 through 90° to its closed position. In this position, the blocking member 34 is located directly in front of the opening 23, thereby preventing most of the gas flow between the HME device 2 and the tube 1. When the patient exhales, therefore, all the exhaled gas passes through the fenestrations 13 in the tube 1 and flows to the vocal folds to enable speech. Inhaled air passes to the lungs in the opposite direction. When the patient has finished talking he rotates the HME unit 30 back to its original, open position.

The arrangement of the present invention can be lightweight, thereby minimizing forces on the patient at the site of the tracheostomy. A rotatable HME unit that enables speech can easily provided for inclusion in some existing HME housings, thereby reducing cost. The HME unit can easily be inserted, removed and replaced where necessary. The present invention also has the advantage that the device can be retained in the speech position without the need for continuous manual intervention. The HME could include HME elements of various different kinds and is not limited to any one kind. The HME does not prevent suctioning taking place.

The HME could include resilient means for returning the HME unit to its open position when released, as shown in FIG. 5. The modified HME unit 30′ shown in FIG. 5 has two integral, springy teeth 131 formed with the frame 31′ at opposite ends. The teeth 131 project longitudinally at the outer edge of the frame 31′ so that they lie against the inside surface of the housing 20′. In this embodiment, the housing 20′ extends a short distance beyond the opposite ends of the HME unit 30′ and has an inwardly-projecting ramp formation 132 moulded into its inner surface at each end to one side of the teeth 131. The HME unit 30′ also has a short peg 133 extending radially outwardly midway along its length on the opposite side of the unit from the teeth 131. The peg 133 locates in a short circumferential slot 134 through the housing 20 and projects outwardly a short distance so that it can be slid manually along the slot to rotate the HME unit 30′ through about 90°. Engagement of the teeth 131 with the ramps 132 urges the HME unit 30′ to the open position with the peg 133 at one end of the slot 134. When the user wishes to speak, he grips the peg 133 and slides it to the opposite end of the slot 134, thereby rotating the HME unit 30′ to the closed position. As the HME unit 30′ rotates, the teeth 131 bend resiliently by engagement with the ramps 132. When the user releases the peg 133, the teeth 131 tend to straighten and, by engagement on the ramps 132, rotate the HME unit 30′ back to its natural position. It will be appreciated that there are many different resilient arrangements that could be used to restore the HME unit back to its original position.

The invention, in some of its aspects, is not confined to HMEs but could be used with other gas-treatment devices such as filters.

1. A gas-treatment device for use with a tracheostomy tube, the device including a housing adapted for connection to the tube, a gas passage in the housing a gas-treatment unit mounted with the housing, characterized in that the gas-treatment unit is displaceable from a first position in which gas can flow from the tube through the gas-treatment unit via the gas passage, to a second position in which the gas passage is substantially blocked preventing flow of gas out of the machine end of the tracheostomy tube.

2. A gas-treatment device according to claim 1, characterized in that the gas-treatment unit is displaceable by rotation.

3. A gas-treatment device according to claim 1, characterized in that the gas-treatment unit remains in the first or second position unless manually displaced.

4. A gas-treatment device according to claim 1, characterized in that the gas-treatment unit includes a projection accessible at an end of the housing by which the gas-treatment unit can be displaced.

5. A gas-treatment device according to claim 1, characterized in that the device includes a resilient member arranged to displace the gas-treatment unit to a first position when released.

6. A gas-treatment device according to claim 1, characterized in that the gas-treatment unit is cylindrical.

7. A gas-treatment device according to claim 1, characterized in that the gas-treatment unit includes an HME element.

8. A gas-treatment device according to claim 6, characterized in that the gas-treatment unit includes an HME element at opposite ends.

9. A gas-treatment device according to claim 7, characterized in that the or each HME element is of a treated paper.

10. A tracheostomy tube assembly including a tracheostomy tube and a gas-treatment device according to claim 1 connected at the machine end of the tube.
